(19) United States
(12) Patent Application Publication
(10) Pub. No.: US 2016/0346305 A1
(43) Pub. Date: Dec. 1, 2016

(54) SWEETENER COMPOSITION FOR PREVENTING AND IMPROVING OBESITY, CONTAINING GLYCOLYSIS INHIBITOR INGREDIENT

(71) Applicant: CJ CHEILJEDANG CORPORATION, Seoul (KR)

(72) Inventors: Young Jae Kim, Seoul (KR); Jin Hee Park, Goyang-si (KR); Min Hae Kim, Incheon (KR); Seong Bo Kim, Seoul (KR); Se Hee Hwang, Seoul (KR); Young Mi Lee, Bucheon-si (KR)

(21) Appl. No.: 15/234,331

(22) Filed: Aug. 11, 2016

Related U.S. Application Data
(63) Continuation of application No. 14/345,156, filed on Mar. 14, 2014.

(30) Foreign Application Priority Data
Sep. 15, 2011 (KR) 10-2011-0092800

(51) Int. Cl. A61K 31/7004; A23L 33/105; A23L 33/00; A23L 27/30; A61K 9/00; A23L 2/60

(52) U.S. Cl. CPC A61K 31/7004; A61K 9/0095; A23L 2/60; A23L 33/30; A23L 27/34; A23L 27/33; A23L 33/105; A23V 2002/00

(57) ABSTRACT

The present invention relates to a sweetener composition for preventing or treating obesity containing, as active ingredients, a glucose or D-fructose absorption inhibiting component and a sugar hydrolysis inhibiting sugar or sugar alcohol.

### CROSS REFERENCE TO RELATED APPLICATIONS

The present application is a continuation application of U.S. application Ser. No. 14/345,156 filed Mar. 14, 2014, which is a U.S. national phase application of PCT/KR2012/007424 filed Sep. 17, 2012, which claims priority to KR Application No. 10-2011-0092800 filed Sep. 15, 2011. U.S. application Ser. No. 14/345,156 is herein incorporated by reference in its entity.

### TECHNICAL FIELD

The present invention relates to a sweetener composition for preventing or treating obesity containing a glucose or D-fructose absorption inhibiting component and a sugar hydrolysis inhibiting sugar or sugar alcohol as active ingredients.

### BACKGROUND ART

Sugar contains sucrose as a main ingredient and is one of representative sweeteners exhibiting sweet taste upon adding to food. Sugar has outstanding sweetness and thus has been considered as one of the most preferred sweeteners which are added to various foods and processed foods to improve the food taste and stimulate appetite. However, recently, as the harmful effects of sugar have been revealed, problems concerning use thereof are being reported. Specifically, excessive sugar consumption is a major cause of tooth decay as well as various lifestyle related diseases such as obesity, diabetes, and the like. For these reasons, there is global demand for an alternative sweetener as a replacement for sugar.

D-psicose is an epimer of D-fructose and is a sub-category of functional sugars known as rare sugars. It is known that D-psicose has a high degree of sweetness equivalent to about 60% to 70% that of sugar and has close to zero calories, and thus is effective in treating adult diseases such as obesity and the like. In addition, D-psicose is also known to have efficacy to prevent and treat diabetes since D-psicose is able to inhibit the absorption of sugars such as glucose, D-fructose and the like. Furthermore, D-psicose is known to have excellent solubility and thus is drawing keep attention for application to foods.

D-psicose has a relatively good sweetness, but has a relatively lower sweetness than that of sugar. In this regard, the use of D-psicose alone as a sweetener for food additives cannot satisfy consumers accustomed to the taste of sugar, thereby hindering market acceptance. In order to overcome such problems stemming from the use of D-psicose alone, namely, in order to achieve sweetness satisfying general consumers while using D-psicose alone, it is inevitable to increase the amount of D-psicose, which can provide excessive thick feeling to foods utilizing D-psicose, thereby causing deterioration in texture of foods.

Therefore, there is a strong need for a sweetener applicable to patients suffering from obesity and inhibiting or capable of preventing obesity using D-psicose having low calories and relatively good sweetness.

As the prior art related to the present invention, there are Korea Patent Publication No. 10-2009-0082403A (published on Jul. 30, 2009), Korea Patent Publication No. 10-2011-0035805A (published on Apr. 6, 2011), Korean Patent No. 10-0779160 B1 (published on Nov. 19, 2007), and the like.

### DISCLOSURE

### Technical Field

The present invention is aimed at providing a sweetener composition for preventing or treating obesity, which comprises a glucose or D-fructose absorption inhibiting component and a sugar hydrolysis inhibiting sugar or sugar alcohol, as active ingredients, wherein the composition has efficacy to prevent or treat obesity while improving quality of sweetness.

### Technical Solution

In accordance with one embodiment of the present invention, a sweetener composition for preventing or treating obesity comprises a glucose or D-fructose absorption inhibiting component and a sugar hydrolysis inhibiting sugar or sugar alcohol as active ingredients.

In accordance with another embodiment of the present invention, the glucose and D-fructose absorption inhibiting component may include D-psicose.

In accordance with a further embodiment of the present invention, the sugar or sugar alcohol may include tagatose, xylose, arabinose, ribose, xylitol, erythritol, or combinations thereof.

In accordance with yet another embodiment of the present invention, the sweetener composition may further include a high sweetness sweetener material.

In accordance with yet another embodiment of the present invention, the high sweetness sweetener material may include steviol glycoside, sucralose, aspartame, Sirailia grosvenoriextract, Glycyrrhiza uralensis Fischer extract, thaumatin, or combinations thereof.

In accordance with yet another embodiment of the present invention, the sugar or sugar alcohol may be present in an amount of 0.01 to 200 times the weight of the glucose and D-fructose absorption inhibiting component, and the high sweetness sweetener material may be present in an amount of 0.001 to 2 times the weight of the glucose and D-fructose absorption inhibiting component.

### Advantageous Effects

The present invention provides a sweetener composition that is effective in preventing or treating obesity by utilizing a glucose or D-fructose absorption inhibiting component and a sugar hydrolysis inhibiting sugar or sugar alcohol, thereby primarily inhibiting hydrolysis of sugars introduced through foods and secondarily inhibiting absorption of digested glucose and D-fructose, which prevents intake of a great quantity of sugars.

In addition, according to another embodiment, the present invention provides a sweetener composition, which includes a glucose or D-fructose absorption inhibiting component and a sugar hydrolysis inhibiting sugar or sugar alcohol in a specific ratio, and thus provides outstanding properties in terms of prevention and treatment of obesity as compared with sweetener compositions prepared in component ratios outside the ranges set forth herein.

According to a further embodiment, the present invention provides a sweetener composition for preventing or treating obesity, which can reduce caloric intake while increasing sweetness and has improved quality of sweetness by adding a specific high sweetness sweetener.

According to yet another embodiment, the present invention provides a sweetener composition for preventing or treating obesity, which exhibits outstanding sweetness, by employing rebaudioside A among the high sweetness sweetener materials, thereby avoiding bitter taste, metallic taste or other characteristics of certain high sweetness sweeteners including steviol glycosides while being naturally derived.

### BRIEF DESCRIPTION OF THE DRAWINGS

FIG. 1 is a graph depicting change of blood glucose level according to Experimental Example 1.

FIG. 2 is a graph depicting change of area under the curve of blood glucose according to Experimental Example 2.

### MODE FOR INVENTION

Hereinafter, the present invention will be described in more detail. Descriptions of details apparent to those skilled in the art having ordinary knowledge in this technical field or relevant fields will be omitted herein.

The present invention provides a sweetener composition for preventing or treating obesity, which contains a glucose or D-fructose absorption inhibiting component and a sugar hydrolysis inhibiting sugar or sugar alcohol as active ingredients.

In accordance with one embodiment, the present invention provides a sweetener composition for preventing or treating obesity, which includes D-psicose as the glucose and D-fructose absorption inhibiting component.

In accordance with another embodiment, the present invention provides a sugar or sugar alcohol sweetener composition for preventing or treating obesity, which includes tagatose, xylose, arabinose, ribose, xylitol, erythritol, or combinations thereof as the sugar hydrolysis inhibiting sugar or sugar alcohol.

Tagatose is a stereoisomer of D-galactose and may be prepared by chemical isomerization of D-galactose using Ca(OH)₂ as a catalyst, or by biotransformation of D-galactose isomerizing D-galactose solutions byan isomerization enzyme.

Tagatose is known as a natural sugar having a high sweetness corresponding to about 90% the sweetness of sucrose and having a low caloric content of about 1.5 kcal per g. Further, tagatose has substantially no side effects and thus is a functional sugar well suited to application to a variety of foods.

Xylose is a natural sugar found in birch, corn and the like and has about 40% the sweetness of sucrose. Xylose is known as one of sweetener materials that can avoid or reduce side effects of sugar upon consumed with sugar. When xylose is consumed together with sugar, it may inhibit the activity of sucrase which is a sugar digestion enzyme, thereby inhibiting the digestion of sugar. As a result, sugar absorption into the body is inhibited and more sugar is discharged from the body undigested.

Arabinose is a natural sugar existing in needle leaf trees and is a component of polysaccharides (hemicellulose, plant rubber, pectin and the like). Arabinose is also found in some bacteria. Arabinose may inhibit the absorption of sugar and specifically inhibit the activity of sucrase, which digests sugar in the small intestine, even when added in an amount of about 3% or so. As a result, arabinose may inhibit sharp rise in blood glucose levels.

Ribose refers to a kind of pentose having five carbon atoms.

Xylitol is a sugar alcohol obtained by reducing xylose extracted from birch, corn and the like. It is known that xylitol is effective in preventing tooth decay due to the pentose structure thereof. Tooth decay refers to a phenomenon that Streptococcus mutans such as Mutans or Sorbinus intake glucose or fructose contained in foods and discharge lactic acid, which erodes the surface of teeth. Such Streptococcus mutans may easily digest hexose, but is not able to digest pentoses such as xylitol. Therefore, xylitol does not produce any acids which may cause tooth decay. Further, Streptococcus mutans that fails to receive any nutrients cannot grow on the tooth surface, thereby preventing dental caries. Furthermore, xylitol has similar sweetness to sugar and produces a refreshing cooling sensation on the palette. For such reasons, xylitol has been utilized as a sweetener in gums, candies and the like.

Erythritol is a kind of sugar alcohol found in lichens, mushrooms, fruits and the like and may be obtained industrially by microbial conversion from glucose by employing yeasts of the genus Aureobasidium.

Yet a further embodiment of the present invention provides a sweetener composition for preventing or treating obesity, which further includes a high sweetness sweetener material.

The high sweetness sweetener material refers to a sweetener material exhibiting high sweetness several times to hundreds of times higher than that of sugar.

The high sweetness sweetener material may include at least one selected from steviol glycoside, sucralose, aspartame, Siraitia grosvenori extract, Glycyrrhiza uralensis Fischer extract or thaumatin, without being limited thereto.

Steviol glycoside refers to a material obtained by processing a water soluble extract of the leaf of Stevia rebaudiana. Preferably, rebaudioside A (Reb A) is used as the steviol glycoside. When rebaudioside A is used as a high sweetness sweetener material, the bitter taste, metallic taste and the like characteristic of high sweetness sweeteners such as steviol glycoside may be avoided, thereby providing a sweetener composition for preventing or treating obesity, which has excellent taste.

When rebaudioside A and tagatose are used simultaneously, there is a synergistic effect that improves quality of sweetness inherent to each substance. For example, although tagatose lacks sweetness persistency, such a drawback of tagatose may be supplemented by rebaudioside A, while the bitterness of rebaudioside A may be overcome by use of tagatose. Therefore, the use of tagatose and rebaudioside A in combination may give a bulky effect, which is heavy feeling of taste in mouth.

Sucralose refers to a synthetic analog of sugar obtained by substituting hydroxyl groups of sugar with chlorine.

Aspartame refers to an amino acid type synthetic sweetener prepared from phenylalanine and aspartic acid.

Siraitia grosvenori extract refers to an extract obtained from fruits of Siraitia grosvenori, which is a perennial herb belonging to the family Cucurbitaceae. The extract used in the present invention is not particularly limited as to extraction method. Any extract prepared by methods known in the technical field of the present invention or similar field may be used.

Glycyrrhiza uralensis Fischer extract refers to an extract obtained from Glycyrrhiza uralensis, which is a perennial herb belonging to genus Cassia. The extract used in the present invention is not particularly limited as to extraction method. Any extract prepared by methods known in the technical field of the present invention or similar field may be used.

Thaumatin refers to a protein type sweetener obtained by extracting fruits of Thaumatococcus daniellii, followed by purifying the extract. Any extract prepared by methods known in the technical field of the present invention or similar field may be used.

According to another embodiment of the present invention, the sweetener composition for preventing or treating obesity contains the sugar or sugar alcohol in an amount of 0.01 times to 200 times, preferably 0.01 times to 100 times, more preferably 0.1 times to 50 times the weight of the glucose and D-fructose absorption inhibiting component.

Further, the sweetener composition for preventing or treating obesity contains the high sweetness sweetener material in an amount of 0.001 times to 2 times, preferably 0.001 times to 1.5 times, more preferably 0.001 times to 1 time the weight of the glucose and D-fructose absorption inhibiting component.

Within this content range, upon ingesting the sweetener, monosaccharide absorption inhibition in the small intestine and carbohydrate digestion inhibition in the small intestine may be suitably exerted, thereby leading to excellent synergistic effects. With this, sharp rise in blood glucose levels after meals may be significantly lowered and the amount of sugar absorbed into the body may be reduced, thereby indicating excellent effect in improving and/or preventing obesity.

Hereinafter, the present invention will be described in more detail with reference to the following Examples, Comparative Examples and Experimental Examples. These examples are provided for illustration only and are not to be in any way construed as limiting the present invention.

### EXAMPLE 1, 2 AND COMPARATIVE EXAMPLE 1 TO 3

### Preparation of Sweetener Composition

Sweetener compositions as listed in Table 1 were prepared. In Comparative Example 1, 5 g of sugar was used. Sweetener compositions of Comparative Examples 2 and 3 and Examples 1 and 2 were prepared by formulating the components as listed in Table 1 such that the compositions exhibited sweetness similar to that of 5 g of sugar.

TABLE 1

| | | Relative | | | | | |
|---|---|---|---|---|---|---|---|
| Raw materials | | Sweetness per g | Com. | Com. | Com. | | |
| (g) | Manufacturer | of raw material | Ex. 1 | Ex. 2 | Ex. 3 | Ex. 1 | Ex. 2 |
| Sugar | CJ Cheiljedang | 1 | 5 | — | — | — | — |
| Erythritol | Zivogreen | 0.63 | — | 8 | — | — | — |
| D-psicose | CJ Cheiljedang | 0.56 | — | — | 9 | 4 | 2.1 |
| Tagatose | CJ Cheiljedang | 0.92 | — | — | — | 3 | 2 |
| Rebaudioside A | GLG | 200 | — | — | — | — | 0.01 |

| | | | | | |
|---|---|---|---|---|---|
| Relative sweetness of sweetener composition | 5 | 5.04 | 5.04 | 5 | 5.02 |

Among the sweetener compositions, erythritol used in Comparative Example 2 corresponds to a material that has substantially zero calories and provides only sweetness without affecting blood glucose level. Rebaudioside A used in Example 2 is a steviol glycoside and is a natural high sweetness sweetener prepared by extracting components having sweet taste from steviol glycosides.

### EXPERIMENTAL EXAMPLE 1

### Measurement of Changes in Blood Glucose Level

(1) Preparation of Test Specimen for Measuring Blood Glucose and Method of Intake

In order to measure changes in blood glucose level after consuming each sweetener composition prepared in Comparative Examples 1 to 3 and Examples 1 and 2, the following experiment was carried out for a normal group having a fasting glycemic index of 100 mg/dL or less and consisting of five males and five females in their twenties to forties.

The sweetener composition intake was conducted by providing subjects with an identical meal and then allowing them to drink coffee containing the sweetener composition.

The meals given to the subjects are as listed in Table 2.

TABLE 2

| Food materials | Amount used (g) |
|---|---|
| Bread | 75 |
| Ham | 20 |
| Lettuce | 20 |
| Strawberry jam | 20 |
| Crabstick | 30 |
| Cheddar cheese | 10 |

As shown in Table 2, the meal given to the subjects consisted of 75 g of bread, 20 g of lettuce, 20 g of strawberry jam, 30 g of crabstick and 10 g of cheddar cheese. As a result of analysis using Korean food composition table (CanPro 3.0, The Korean Nutrition Society), it was found that the meal had a total caloric content of 356.4 kcal, which consisted of 59.57% of sugar, 18.14% of protein and 22.27% of lipid.

After the meal, the subjects drank coffee made by mixing 1.6 g of sugar free coffee and the sweetener composition prepared in Comparative Examples 1 to 3 and Examples 1 and 2 in 200 g of hot water.

The coffee composition given to the subjects is listed in Table 3.

TABLE 3

| | | Comparative | Comparative | Comparative | | |
|---|---|---|---|---|---|---|
| Materials (g) | Manufacturer | Example 1 | Example 2 | Example 3 | Example 1 | Example 2 |
| Coffee | Dongsuh Food | 1.6 | 1.6 | 1.6 | 1.6 | 1.6 |
| Sugar | CJ Cheiljedang | 5 | — | — | — | — |
| Erythritol | Zivogreen | — | 8 | — | — | — |
| D-psicose | CJ Cheiljedang | — | — | 9 | 4 | 2.1 |
| Tagatose | CJ Cheiljedang | — | — | — | 3 | 2 |
| Rebaudioside A | GLG | — | — | — | — | 0.01 |

In order to measure changes in blood glucose after the meal, blood glucose level before meal was checked, followed by providing the subjects with the meal and then coffee was given. Blood glucose was measured for 2 hours at intervals of 30 minutes.

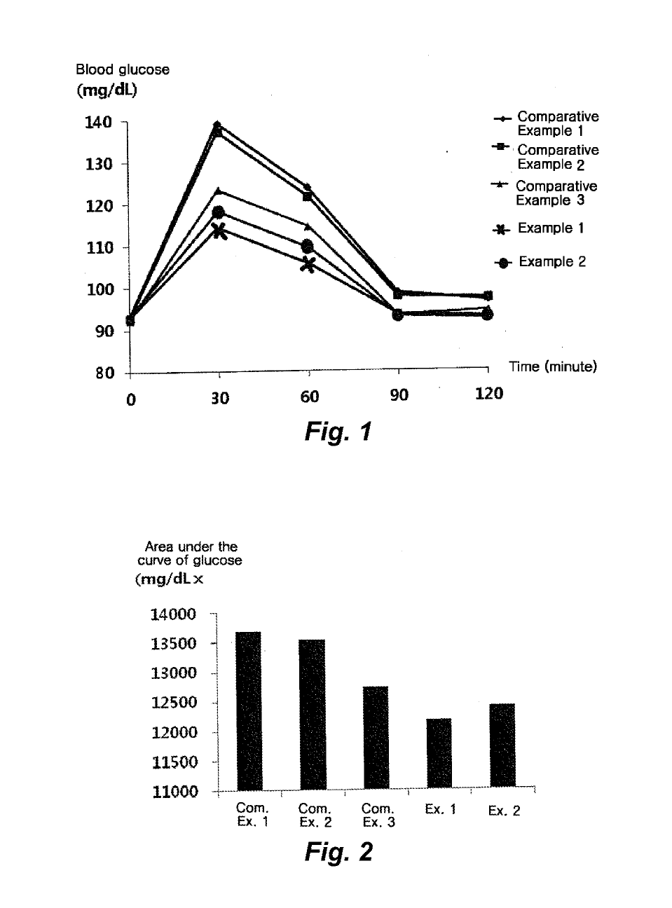

The changes in blood glucose level after meal and coffee intake are summarized in Table 4 (see FIG. 1).

TABLE 4

| | | Time (min) | | | | |
|---|---|---|---|---|---|---|
| | | 0 (before | | | | |
| | | meal) | 30 | 60 | 90 | 120 |
| Blood | Comparative | 93.1 | 139.1 | 123.7 | 98.5 | 96.7 |
| glucose | Example 1 | | | | | |
| (mg/dL) | Comparative | 92.8 | 137.1 | 121.6 | 97.7 | 97.2 |
| | Example 2 | | | | | |
| | Comparative | 92.4 | 123.3 | 114.5 | 93.1 | 94.1 |
| | Example 3 | | | | | |
| | Example 1 | 92.9 | *114.3 | *105.5 | *93.4 | 92.8 |
| | Example 2 | 93.2 | *118.3 | *109.5 | *92.9 | 92.4 |

*corresponds to time zones in Examples 1 and 2 indicating significant difference of p < 0.01 or less in blood glucose as compared with Comparative Example 1 (sugar).

### EXPERIMENTAL EXAMPLE 2

### Measurement of Changes in Area Under the Curve of Blood Glucose

The same experiment as in Experimental Example 1 was performed for 2 hours at an interval of 30 minutes to measure G-AUC (Glucose-Area under curve) of the subjects.

The results of G-AUC are summarized in Table 5.

TABLE 5

| | | Time (min) | | | |
|---|---|---|---|---|---|
| | | 30 | 60 | 90 | 120 |
| G-AUC | Comparative | 3483 | 7425 | 10758 | 13686 |
| (mg/dL × min) | Example 1 | | | | |
| | Comparative | 3449 | 7329 | 10619 | 13542 |
| | Example 2 | | | | |
| | Comparative | 3236 | 6803 | 9917 | 12725 |
| | Example 3 | | | | |
| | Example 1 | 3108 | 6405 | 9389 | 12182 |
| | Example 2 | 3173 | 6590 | 9626 | 12405 |

As shown in Table 5, both Examples 1 and 2 showed significant decrease in G-AUC for 120 minutes as compared with Comparative Example 1 (see FIG. 2).

As can be seen from the results of Experimental Examples 1 and 2, increase in post-meal blood glucose persisted for about 60 minutes and 60 minutes post-meal, the blood glucose level showed a tendency to return to fasting blood glucose level.

When the subjects drank coffee containing the sweetener composition after the meal, increase in blood glucose to 60 minutes after drinking coffee was high in order of Comparative Example 1, Comparative Example 2, Comparative Example 3, Example 2 and Example 1.

Obesity is most likely to occur when the amount of energy absorbed from various components of foods is higher than the energy consumed from activity and is accumulated.

Comparative Example 1 showed the highest increase in blood glucose resulting from the combined increase in blood glucose due to the meal and the coffee containing sugar. Comparative Example 2 showed increase in blood glucose due to meal only. Comparative Example 3 showed that D-psicose has an effect of lowering increase in blood glucose after the meal through inhibition of monosaccharide absorption in the small intestine.

Examples 1 and 2 appeared to show considerably low absorption of blood glucose as compared with Comparative Examples showing sharp rise in blood glucose after the meal. It appears that such result was caused by rebaudioside A used as the high sweetness sweetener material to increase sweetness, thereby decreasing the amount of tagatose and D-psicose used.

### EXPERIMENTAL EXAMPLE 3

### Sensory Evaluation of Sweetener Composition

In order to evaluate sensory perception of the sweetener compositions prepared in Examples 1 and 2, a sensory evaluation test was performed on 25 adult males and females in their twenties to fifties using coffee containing sugar prepared in the same manner as in Experimental Example 1 (corresponding to Comparative Example 1 of Experimental Example 1) and coffee each containing the sweetener composition of Examples 1 and 2.

The sensory analysis was evaluated on a scale of 1 to 5. The results of the sensory analysis are summarized in Table 6.

TABLE 6

| | General | Color | General flavor | Taste | Aftertaste |
|---|---|---|---|---|---|
| | accept- | accept- | accept- | accept- | accept- |
| | ability | ability | ability | ability | ability |
| Coffee | 3.30 | 3.62 | 3.52 | 3.34 | 3.22 |
| containing | | | | | |
| sugar | | | | | |
| Example 1 | 3.15 | 3.60 | 3.35 | 3.28 | 3.22 |
| Example 2 | 3.32 | 3.55 | 3.50 | 3.35 | 3.25 |

As can be seen from the results of Experimental Examples 1 to 3, it is determined that the sweetener composition of the present invention has excellent synergistic effect of suitably exerted monosaccharide absorption inhibition in the small intestine due to D-psicose and carbohydrate digest inhibition in the small intestine due to tagatose, thereby preventing sharp increase in blood glucose after the mean while reducing the amount of sugar absorbed into the body. For these reasons, the composition has excellent effects of improving and/or preventing obesity and thus corresponds to the sweetener material for preventing or treating obesity.

1. A method for lowering increase of blood glucose level, comprising administering a sweet composition containing D-psicose and tagatose as active ingredients, to the subject.

2. The method as claimed in claim 1, wherein the sweetener composition lowers increase of blood glucose level after meal.

3. The method as claimed in claims 1, wherein the sweet composition further comprises a high-intensity sweetener.

4. The method as claimed in claim 3, wherein the sweet composition comprises at least one high-intensity sweetener selected from the group consisting of steviol glycoside, sucralose, aspartame, Siraitia grosvenori extract, Glycyrrhiza uralensis Fischer extract, and thaumatin.

5. The method as claimed in claim 3, wherein the tagatose is present in an amount of 0.01 to 200 times based on the weight of D-psicose, and the high-intensity sweetener is present in an amount of 0.001 to 2 times based on the weight of D-psicose.

6. The method as claimed in claim 1, wherein the sweet composition is provided as a beverage.

7. The method as claimed in claim 1, wherein the sweet composition is administered after a meal.

8. The method as claimed in claim 2, wherein the after meal means within 1 hour after a meal.
